(19) United States
(12) Patent Application Publication
(10) Pub. No.: US 2022/0235387 A1
(43) Pub. Date: Jul. 28, 2022

(54) Method for Preparing Bacterial Cellulose Membrane Using Enzymatic Soybean Hydrolysate

(71) Applicant: Northeast Agricultural University, Harbin (CN)

(72) Inventors: Yang Li, Harbin (CN); Baokun Qi, Harbin (CN); Li Huang, Harbin (CN); Liang Cao, Harbin (CN)

(21) Appl. No.: 17/717,211

(22) Filed: Apr. 11, 2022

Related U.S. Application Data
(63) Continuation of application No. PCT/CN2019/115679, filed on Nov. 5, 2019.

(51) Int. Cl. C12P 19/04; A23J 3/34; B01D 21/26

(52) U.S. Cl. CPC C12P 19/04; B01D 21/262; A23J 3/346

(57) ABSTRACT

The disclosure provides a method for preparing a bacterial cellulose membrane using an enzymatic soybean hydrolysate, comprising the following steps: (1) preparation of an enzymatic soybean hydrolysate medium; (2) preparation of a crude bacterial cellulose membrane; and (3) purification of a bacterial cellulose membrane. The method of the disclosure rationally uses the residual waste liquid obtained from enzymatic preparation of soybean oil, and without acid hydrolysis treatment of a medium, the bacterial cellulose synthesized by bacterial strains directly using an enzymatic soybean hydrolysate has a greater amount, finer and denser microfibers, and a higher maximum thermal degradation temperature.

### TECHNICAL FIELD

The disclosure belongs to the technical field of preparation of bacterial cellulose membranes, and particularly relates to a method for preparing a bacterial cellulose membrane using an enzymatic soybean hydrolysate.

### BACKGROUND

Bacterial cellulose (BC) refers to a general term for cellulose synthesized by certain microorganisms such as Acetobacter, Agrobacterium, Rhizobium and Sarcina under different conditions. The bacterial cellulose forms a unique textured structure, and due to a “nano effect”, the bacterial cellulose has the characteristics including high water absorption and high water retention, high permeability to liquid and gas, high wet strength, and especially in-situ processing and molding in a wet state. The high purity and excellent properties of nano-effect make bacterial cellulose fibers widely used in special fields.

At present, there are studies on the use of various industrial and agricultural wastes as mediums for preparing bacterial cellulose, such as soybean molasses produced by processing protein concentrates and molasses produced by processing sucrose or sugar beets. Because bacterial strains for synthesizing bacterial cellulose use single glucose in the cellulose synthesis process, high-strength acid hydrolysis (hydrochloric acid, nitric acid, sulfuric acid, etc.) is required before the molasses is used as a medium, so as to increase the fermentation rate of the bacterial strains. In addition, the whey water (lactalbumin and albumin) produced by the production of soybean protein isolate is also used, which is not conducive to the use of the bacterial strains.

Enzymatic extraction of vegetable oil is an emerging oil extraction technology developed in the 1970s. As an emerging vegetable oil extraction technology, on the basis of mechanical crushing, enzymolysis is performed on oil tissues and complexes such as lipoproteins and lipopolysaccharides to free the oil. The residual water phase obtained from enzymatic extraction of vegetable oil still contains many valuable components, the waste of resources may be caused if the residual water phase is directly discarded, and thus, the residual water phase may be used.

### SUMMARY

In order to solve the problem that the residual waste liquid obtained from enzymatic preparation of soybean oil can not be rationally used, the disclosure provides a method for preparing a bacterial cellulose membrane using an enzymatic soybean hydrolysate. Specific steps are as follows:

(1) preparation of an enzymatic soybean hydrolysate medium: taking a residual waste liquid obtained from enzymatic preparation of soybean oil as a raw liquid, adjusting a pH to 4.3-4.6, and removing impurities to obtain a medium;

(2) preparation of a crude bacterial cellulose membrane: inoculating a fermentation strain into the enzymatic soybean hydrolysate liquid medium obtained in step (1) for fermentation at a fermentation temperature of 25-32° C. for a fermentation time of 8-15 days to obtain a crude bacterial cellulose membrane; and

(3) purification of a bacterial cellulose membrane: boiling the crude bacterial cellulose membrane obtained in step (2) in boiling water for 10-30 min, then boiling the bacterial cellulose membrane with 0.1-1 M sodium hydroxide for 20-60 min, soaking the bacterial cellulose membrane in water to adjust the pH to neutral, and washing the bacterial cellulose membrane with the changed water until the cellulose membrane is transparent.

Preferably, the waste liquid in step (1) is prepared by the following method: crushing soybeans, pressing the crushed soybeans into flakes, adding water for blending soybean flour mucilage, then performing extrusion and puffing treatment, mixing the obtained soybean flour with water to obtain a soybean flour liquid, performing enzymolysis on the soybean flour liquid with a complex enzyme, and performing three-phase separation on an enzymolysis liquid to obtain a hydrolysate.

Preferably, the process of removing impurities in step (1) is to use a filter sieve with 300-400 meshes to filter the raw liquid to remove impurities.

Preferably, the fermentation strain in step (2) is Kombucha.

Preferably, an inoculation concentration of the fermentation strain in step (2) is 6×10⁶-6×10⁸ cfu/ml.

Preferably, temperatures for the extrusion and puffing are 40-60° C. for the first stage, 60-80° C. for the second stage, 80-95° C. for the third stage, and 95-105° C. for the fourth stage.

Preferably, the temperatures for the extrusion and puffing are 48° C. for the first stage, 72° C. for the second stage, 86° C. for the third stage, and 102° C. for the fourth stage.

Preferably, the enzymolysis with a complex enzyme refers to enzymolysis on the soybean flour liquid by a mixed enzyme of alkaline protease with a mass fraction of 4%-8% of soybean flour and flavourzyme with a mass fraction of 1%-4% of soybean flour.

Preferably, the enzymolysis with a complex enzyme is enzymolysis on the soybean flour liquid by a mixed enzyme of alkaline protease with a mass fraction of 5% of soybean flour and flavourzyme with a mass fraction of 2% of soybean flour.

Preferably, the three-phase separation is performed by a three-phase horizontal separator at a centrifugal rotational speed of 4300-4500 r under the condition of centrifugation for 10-20 s.

Beneficial Effect

The method of the disclosure rationally uses the residual waste liquid obtained from enzymatic preparation of soybean oil, and without acid hydrolysis treatment of a medium, the bacterial cellulose synthesized by bacterial strains directly using an enzymatic soybean hydrolysate has a greater amount, finer and denser microfibers, and a higher maximum thermal degradation temperature.

### BRIEF DESCRIPTION OF FIGURES

FIG. 1 shows three-phase horizontal separation results, wherein a shows a separation result in Example 1, b shows a separation result in Example 2, and c shows a separation result in Example 3.

FIG. 2 shows a scanning electron microscope (SEM) observation result of a bacterial cellulose obtained in Example 1.

FIG. 3 shows an SEM observation result of a bacterial cellulose obtained in Example 4.

FIG. 4 shows diameter counting results of the bacterial cellulose obtained in Example 1.

FIG. 5 shows diameter counting results of the bacterial cellulose obtained in Example 4.

FIG. 6 shows an atomic force microscope (AFM) observation result of the bacterial cellulose obtained in Example 1.

FIG. 7 shows an AFM observation result of the bacterial cellulose obtained in Example 4.

FIG. 8 shows thermal degradation temperature results of the bacterial cellulose obtained in Example 1.

FIG. 9 shows thermal degradation temperature results of the bacterial cellulose obtained in Example 4.

### DETAILED DESCRIPTION

The disclosure will be further described below in conjunction with specific examples, but the disclosure is not limited by the examples.

Unless otherwise specified, the materials, reagents, methods and instruments used in the following examples are conventional materials, reagents, methods and instruments in the art, which can be obtained by those skilled in the art through commercial channels. Kombucha is cultivated and produced by Probiotics Family Kombucha Biological Cultivation Co., Ltd.

### Example 1: Method for Preparing a Bacterial Cellulose Membrane Using an Enzymatic Soybean Hydrolysate

The method for preparing a bacterial cellulose membrane using an enzymatic soybean hydrolysate in this example includes the following specific steps:

(1) Preparation of an enzymatic soybean hydrolysate medium:

the enzymatic soybean hydrolysate is a residual waste liquid obtained from enzymatic preparation of soybean oil, and is obtained from soybeans by crushing, flake pressing, blending, extrusion and puffing, pulverizing and sieving, complex enzymolysis and three-phase separation, and specific obtaining steps are as follows:

1) soybeans are crushed into 5 pieces;

2) the crushed soybeans are pressed into 3 mm thick soybean flakes;

3) the soybean flakes obtained in step 2) are mixed with water with a mass fraction of 10.5% to obtain soybean flour mucilage;

4) extrusion and puffing are performed on the soybean flour mucilage obtained in step 3), and puffing temperatures are 48° C. for the first stage, 72° C. for the second stage, 86° C. for the third stage, and 102° C. for the fourth stage;

5) the soybean flour after extrusion and puffing in step 4) is pulverized and then sieved with a 70-mesh sieve, and the sieved soybean flour is mixed with water in a proportion of 1 g:6.5 ml to prepare a soybean flour liquid;

6) alkaline protease with a mass fraction of 5% of soybean flour and flavourzyme with a mass fraction of 2% of soybean flour are mixed with the prepared soybean flour liquid, hydrolysis is performed for 5 h at 55° C., and enzyme deactivation is performed for 3 min at 94° C.; and

7) centrifugation is performed for 16 s by a three-phase horizontal separator at 4400 rad/min to obtain a hydrolysate, a three-phase horizontal separation result is shown in a in FIG. 1, an enzymatic soybean hydrolysate is at the undermost layer, the hydrolysate is sterilized for 19 min at 121° C., and the sterilized hydrolysate is filtered by a 300-mesh filter sieve to remove the precipitated impurities to obtain the enzymatic soybean hydrolysate.

A pH of the enzymatic soybean hydrolysate obtained in the above process is adjusted to 4.5, the enzymatic soybean hydrolysate is filtered by the 300-mesh filter sieve to remove impurities, and the filtrate is sterilized to obtain an enzymatic soybean hydrolysate liquid medium.

(2) Preparation of a Crude Bacterial Cellulose Membrane:

1) a standard HS medium is prepared, components of the medium are glucose (20 g/L), yeast extract powder (5 g/L), peptone (5 g/L), citric acid (1.15 g/L) and disodium hydrogen phosphate (2.7 g/L), and a pH is 6.0;

2) Kombucha with a volume fraction of 6% is inoculated into the HS medium in step 1) and cultured for 24 h at a culture temperature of 28° C. to complete activation and rejuvenation of the bacterial strain;

3) a seed liquid is prepared, and components of the seed liquid are glucose with a mass concentration of 40 g/L and black tea with a mass concentration of 5 g/L; and

4) the Kombucha after activation in step 2) is inoculated into the seed liquid, an inoculation volume is 6% of a volume of the seed liquid, and culture is performed for 24 h at 28° C. until the concentration of the bacterial strain is 10⁷ cfu/ml.

The above bacterial strain is inoculated into the enzymatic soybean hydrolysate liquid medium obtained in step (1) for fermentation in an inoculation volume fraction of 6% and at a fermentation temperature of 28° C. for a fermentation time of 13 days to obtain a crude bacterial cellulose membrane.

(3) Purification of a Bacterial Cellulose Membrane:

the cellulose membrane obtained in step (2) is boiled in boiling water for 20 min and then boiled with 0.2 M sodium hydroxide for 40 min, the cellulose membrane is soaked in water to adjust the pH to neutral, and the cellulose membrane is washed with the changed water until the cellulose membrane is transparent.

### Example 2: Method for Preparing a Bacterial Cellulose Membrane Using an Enzymatic Soybean Hydrolysate

The method for preparing a bacterial cellulose membrane using an enzymatic soybean hydrolysate in this example includes the following specific steps:

(1) Preparation of an Enzymatic Soybean Hydrolysate Medium:

the enzymatic soybean hydrolysate is a residual waste liquid obtained from enzymatic preparation of soybean oil, and is obtained from soybeans by crushing, flake pressing, blending, extrusion and puffing, pulverizing and sieving, complex enzymolysis and three-phase separation, and specific obtaining steps are as follows:

1) soybeans are crushed into 4 pieces;

2) the crushed soybeans are pressed into 2 mm thick soybean flakes;

3) the soybean flakes obtained in step 2) are mixed with water with a mass fraction of 10% to obtain soybean flour mucilage;

4) extrusion and puffing are performed on the soybean flour mucilage obtained in step 3), and puffing temperatures are 40° C. for the first stage, 60° C. for the second stage, 80° C. for the third stage, and 95° C. for the fourth stage;

5) the soybean flour after extrusion and puffing in step 4) is pulverized and then sieved with a 60-mesh sieve, and the sieved soybean flour is mixed with water in a proportion of 1 g:6 ml to prepare a soybean flour liquid;

6) alkaline protease with a mass fraction of 4% of soybean flour and flavourzyme with a mass fraction of 1% of soybean flour are mixed with the prepared soybean flour liquid, hydrolysis is performed for 2 h at 50° C., and enzyme deactivation is performed for 3 min at 90° C.; and

7) centrifugation is performed for 10 s by a three-phase horizontal separator at 4300 rad/min to obtain a hydrolysate, a three-phase horizontal separation result is shown in b in FIG. 1, an enzymatic soybean hydrolysate is at the undermost layer, the hydrolysate is sterilized for 18 min at 121° C., and the sterilized hydrolysate is filtered by a 300-mesh filter sieve to remove the precipitated impurities to obtain the enzymatic soybean hydrolysate.

A pH of the enzymatic soybean hydrolysate obtained in the above process is adjusted to 4.3, the enzymatic soybean hydrolysate is filtered by the 300-mesh filter sieve to remove the precipitated impurities, and the filtrate is sterilized to obtain an enzymatic soybean hydrolysate liquid medium.

(2) Preparation of a Crude Bacterial Cellulose Membrane:

1) a standard HS medium is prepared, components of the medium are glucose (20 g/L), yeast extract powder (5 g/L), peptone (5 g/L), citric acid (1.15 g/L) and disodium hydrogen phosphate (2.7 g/L), and a pH is 6.0;

2) Kombucha with a volume fraction of 6% is inoculated into the HS medium in step 1) and cultured for 20 h at a culture temperature of 25° C. to complete activation and rejuvenation of the bacterial strain;

3) a seed liquid is prepared, and components of the seed liquid are glucose with a mass concentration of 20 g/L and black tea with a mass concentration of 3 g/L; and

4) the Kombucha after activation in step 2) is inoculated into the seed liquid, an inoculation volume is 6% of a volume of the seed liquid, and culture is performed for 20 h at 25° C. until the concentration of the bacterial strain is 10⁶ cfu/ml.

The above bacterial strain is inoculated into the enzymatic soybean hydrolysate liquid medium obtained in step (1) for fermentation in an inoculation volume fraction of 6% and at a fermentation temperature of 25° C. for a fermentation time of 8 days to obtain a crude bacterial cellulose membrane.

(3) Purification of a Bacterial Cellulose Membrane:

the cellulose membrane obtained in step (4) is boiled in boiling water for 10 min and then boiled with 0.1 M sodium hydroxide for 20 min, the cellulose membrane is soaked in water to adjust the pH to neutral, and the cellulose membrane is washed with the changed water until the cellulose membrane is transparent.

### Example 3: Method for Preparing a Bacterial Cellulose Membrane Using an Enzymatic Soybean Hydrolysate

The method for preparing a bacterial cellulose membrane using an enzymatic soybean hydrolysate in this example includes the following specific steps:

(1) Preparation of an Enzymatic Soybean Hydrolysate Medium:

the enzymatic soybean hydrolysate is a residual waste liquid obtained from enzymatic preparation of soybean oil, and is obtained from soybeans by crushing, flake pressing, blending, extrusion and puffing, pulverizing and sieving, complex enzymolysis and three-phase separation, and specific obtaining steps are as follows:

1) soybeans are crushed into 6 pieces;

2) the crushed soybeans are pressed into 4 mm thick soybean flakes;

3) the soybean flakes obtained in step 2) are mixed with water with a mass fraction of 11% to obtain soybean flour mucilage;

4) extrusion and puffing are performed on the soybean flour mucilage obtained in step 3), and puffing temperatures are 60° C. for the first stage, 80° C. for the second stage, 95° C. for the third stage, and 105° C. for the fourth stage;

5) the soybean flour after extrusion and puffing in step 4) is pulverized and then sieved with an 80-mesh sieve, and the sieved soybean flour is mixed with water in a proportion of 1 g:7 ml to prepare a soybean flour liquid;

6) alkaline protease with a mass fraction of 8% of soybean flour and flavourzyme with a mass fraction of 4% of soybean flour are mixed with the prepared soybean flour liquid, hydrolysis is performed for 6 h at 60° C., and enzyme deactivation is performed for 3 min at 90° C.; and

7) centrifugation is performed for 20 s by a three-phase horizontal separator at 4500 rad/min to obtain a hydrolysate, a three-phase horizontal separation result is shown in c in FIG. 1, an enzymatic soybean hydrolysate is at the undermost layer, the hydrolysate is sterilized for 20 min at 121° C., and the sterilized hydrolysate is filtered by a 400-mesh filter sieve to remove the precipitated impurities to obtain the enzymatic soybean hydrolysate.

A pH of the enzymatic soybean hydrolysate obtained in the above process is adjusted to 4.6, the enzymatic soybean hydrolysate is filtered by the 400-mesh filter sieve to remove the precipitated impurities, and the filtrate is sterilized to obtain an enzymatic soybean hydrolysate liquid medium.

(2) Preparation of a Crude Bacterial Cellulose Membrane:

1) a standard HS medium is prepared, components of the medium are glucose (20 g/L), yeast extract powder (5 g/L), peptone (5 g/L), citric acid (1.15 g/L) and disodium hydrogen phosphate (2.7 g/L), and a pH is 6.0;

2) Kombucha with a volume fraction of 6% is inoculated into the HS medium in step 1) and cultured for 48 h at a culture temperature of 32° C. to complete activation and rejuvenation of the bacterial strain;

3) a seed liquid is prepared, and components of the seed liquid are glucose with a mass concentration of 60 g/L and black tea with a mass concentration of 7 g/L; and

4) the Kombucha after activation in step 2) is inoculated into the seed liquid, an inoculation volume is 6% of a volume of the seed liquid, and culture is performed for 48 h at 32° C. until the concentration of the bacterial strain is 10⁸ cfu/ml.

The above bacterial strain is inoculated into the enzymatic soybean hydrolysate liquid medium obtained in step (1) for fermentation in an inoculation volume fraction of 6% and at a fermentation temperature of 32° C. for a fermentation time of 15 days to obtain a crude bacterial cellulose membrane.

(3) Purification of a Bacterial Cellulose Membrane:

the cellulose membrane obtained in step (4) is boiled in boiling water for 30 min and then boiled with 1 M sodium hydroxide for 60 min, the cellulose membrane is soaked in water to adjust the pH to neutral, and the cellulose membrane is washed with the changed water until the cellulose membrane is transparent.

### Example 4: Comparative Example of Example 1

The same as Example 1, the difference is that in preparation of a crude bacterial cellulose membrane in step (2) in this Example, a fermentation medium used in 4) also adopts a standard HS medium to prepare a bacterial cellulose membrane.

By comparing the bacterial cellulose synthesized in Example 1 with the bacterial cellulose synthesized in Example 4, the yield of the cellulose prepared by the enzymatic soybean hydrolysate medium in Example 1 is 1.78 g/L, the yield of the cellulose prepared by the standard HS medium in Example 4 is 1.25 g/L, and thus, the yield is increased by 30.0%.

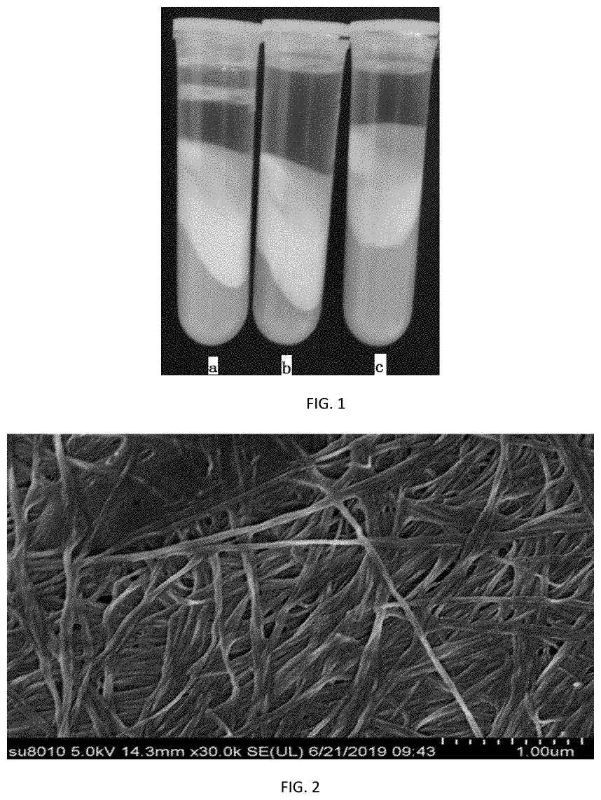

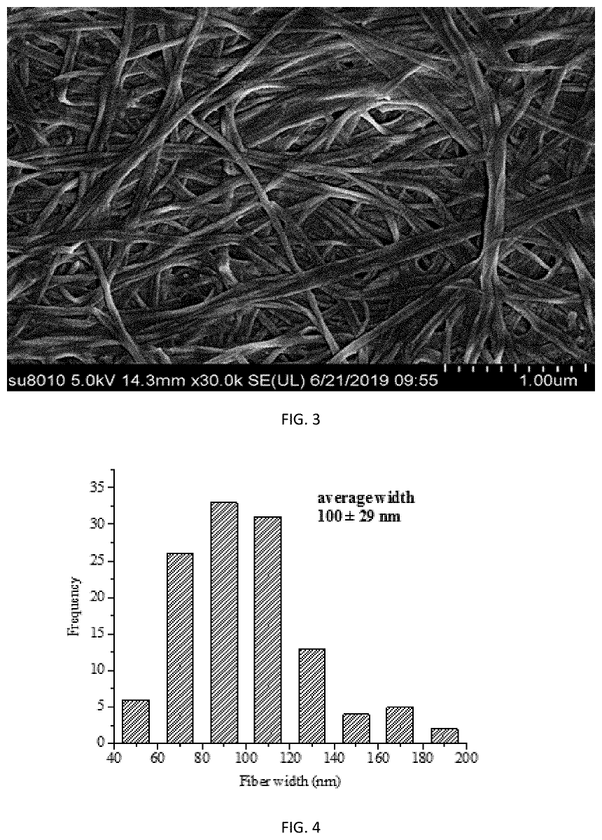

The surface morphology of freeze-dried bacterial cellulose is observed by a scanning electron microscope (SEM, SU8010, HITACHI), and the results show that the bacterial cellulose is composed of rod-shaped nanofibers and forms a porous three-dimensional net structure. The result obtained by the SEM in Example 1 is shown in FIG. 2, and the result of Example 4 is shown in FIG. 3. By comparing the two figures, it can be known that the fibers produced by the standard HS medium in Example 4 are slightly thicker, and the bacterial fibers produced by the enzymatic soybean hydrolysate medium in Example 1 are slightly thinner. The type of a surface medium affects the morphological properties of fibers in the bacterial cellulose.

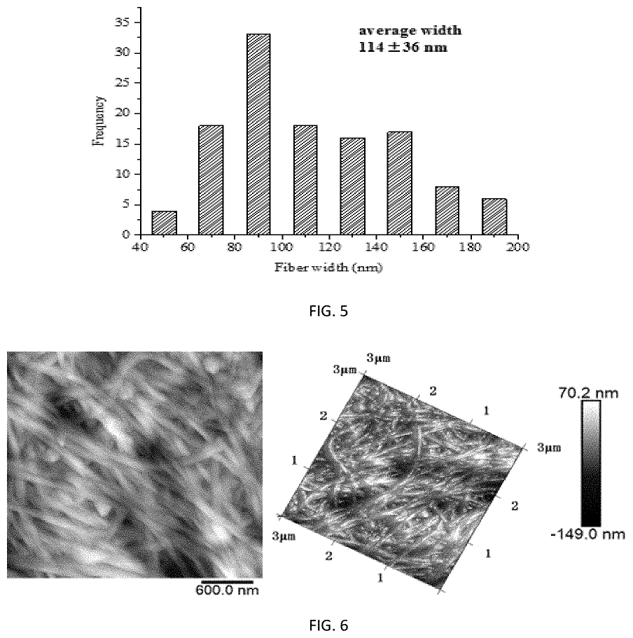

The diameters of the cellulose obtained in Example 1 and Example 4 are counted. The counting results of Example 1 are shown in FIG. 4, and the counting results of Example 4 are shown in FIG. 5. The fiber diameters of the cellulose in FIG. 4 and FIG. 5 are distributed between 40 nm and 200 nm. Comparing the fiber diameters of the bacterial cellulose, the fiber diameter obtained by the enzymatic soybean hydrolysate medium in Example 1 is 100 nm, and the average fiber diameter obtained by the standard HS medium in Example 4 is 114 nm, so that the microfibers of the bacterial cellulose obtained by the enzymatic soybean hydrolysate medium in Example 1 are finer and denser.

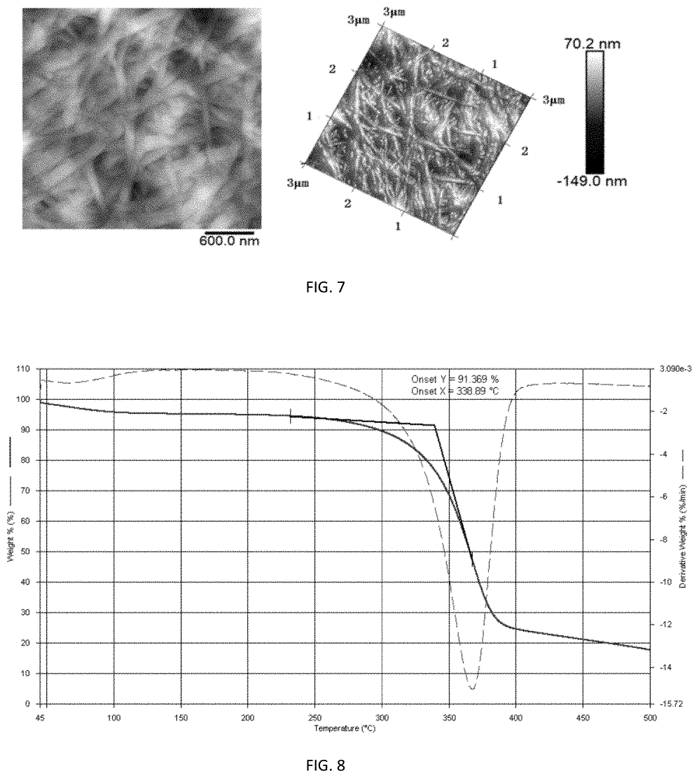

An atomic force microscope (AFM) can be used for observing the microscopic morphology and microscopic details of the surface of the bacterial cellulose, and more clearly observing the dense and aggregated typical structure of the freeze-dried bacterial cellulose. By the observation of the AFM, a nano-scale net structure is shown, and the microfibers of the bacterial cellulose are tightly packed and arranged irregularly. By the observation of the AFM (Bruker, Germany), the results of the three-dimensional structure of the cellulose obtained in Example 1 are shown in FIG. 6, and the results of the three-dimensional structure of the cellulose obtained in Example 4 are shown in FIG. 7. The diameter widths of the microfibers of the bacterial cellulose produced by the standard HS medium and the enzymatic soybean hydrolysate medium in Example 1 are different, and the diameters of the microfibers formed by the standard HS medium are wider, which is consistent with the results observed by the SEM.

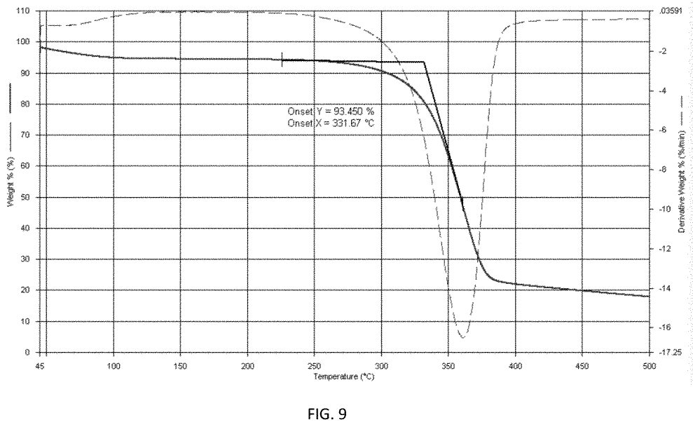

The maximum thermal degradation temperature is analyzed by a thermal gravimetric analyzer (Pyris 6 TGA, Perkin Elmer Co., Ltd. USA). The results of Example 1 are shown in FIG. 8, and the results of Example 4 are shown in FIG. 9. The bacterial cellulose exhibits two different thermal degradation stages. The first thermal degradation stage occurs at 90° C. to 100° C. and is mainly reflected in the absorbed moisture content on the surface and the loss of interlayer coordinated water molecules. The second thermal degradation stage occurs at 300° C. to 400° C., and is reflected in thermal degradation and cracking of bacterial cellulose skeletons, and finally, the bacterial cellulose is decomposed into water, carbon dioxide, etc. The thermal degradation rate temperatures of the bacterial cellulose produced by the standard HS medium in Example 4 and the enzymatic soybean hydrolysate medium in Example 1 are 331.67° C. and 338.89° C. respectively. The thermal degradation rate temperature is increased by 7.22° C.

The yield, morphology, diameter and thermal degradation temperature of the cellulose obtained in Example 2 and Example 3 are similar to those in Example 1.

Although the disclosure has been disclosed above with preferred examples, it is not intended to limit the disclosure. Anyone familiar with this technology can make various changes and modifications without departing from the spirit and scope of the disclosure, and therefore, the protection scope of the disclosure should be defined by the claims.

What is claimed is:

1. A method for preparing a bacterial cellulose membrane using an enzymatic soybean hydrolysate, comprising the following steps:
(1) preparing an enzymatic soybean hydrolysate medium: taking a residual waste liquid obtained from enzymatic preparation of soybean oil as a raw liquid, adjusting its pH to 4.3-4.6, and removing impurities to obtain a medium;
(2) preparing a crude bacterial cellulose membrane: inoculating a fermentation strain into the enzymatic soybean hydrolysate liquid medium obtained in step (1) for fermentation at a fermentation temperature of 25-32° C. for a fermentation time of 8-15 days to obtain a crude form of the bacterial cellulose membrane; and
(3) purifying the bacterial cellulose membrane: boiling the crude form of the bacterial cellulose membrane obtained in step (2) in boiling water for 10-30 min, then boiling with 0.1-1 M sodium hydroxide for 20-60 min, soaking in water to adjust the pH to neutral, and washing the bacterial cellulose membrane with water until the cellulose membrane is transparent.

2. The method according to claim 1, wherein the waste liquid in step (1) is prepared by the following method: crushing soybeans, pressing the crushed soybeans into flakes, adding water for blending soybean flour mucilage, then performing extrusion and puffing treatment, mixing the obtained soybean flour with water to obtain a soybean flour liquid, performing enzymolysis on the soybean flour liquid with a complex enzyme, and performing three-phase separation on an enzymolysis liquid to obtain a hydrolysate.

3. The method according to claim 1, wherein the process of removing impurities in step (1) is to use a filter sieve with 300-400 meshes to filter the raw liquid to remove impurities.

4. The method according to claim 1, wherein the fermentation strain in step (2) is Kombucha.

5. The method according to claim 1, wherein an inoculation concentration of the fermentation strain in step (2) is 6×10⁶-6×10⁸ cfu/ml.

6. The method according to claim 2, wherein temperatures for the extrusion and puffing are 40-60° C. for a first stage, 60-80° C. for a second stage, 80-95° C. for a third stage, and 95-105° C. for the fourth stage.

7. The method according to claim 6, wherein the temperatures for the extrusion and puffing are 48° C. for the first stage, 72° C. for the second stage, 86° C. for the third stage, and 102° C. for the fourth stage.

8. The method according to claim 2, wherein the enzymolysis with a complex enzyme refers to enzymolysis on the soybean flour liquid by a mixed enzyme of alkaline protease with a mass fraction of 4%-8% of soybean flour and flavourzyme with a mass fraction of 1%-4% of soybean flour.

9. The method according to claim 8, wherein the enzymolysis with a complex enzyme is enzymolysis on the soybean flour liquid by a mixed enzyme of alkaline protease with a mass fraction of 5% of soybean flour and flavourzyme with a mass fraction of 2% of soybean flour.

10. The method according to claim 2, wherein the three-phase separation is performed by a three-phase horizontal separator at a centrifugal rotational speed of 4300-4500 r under the condition of centrifugation for 10-20 s.
